(19) United States
(12) Patent Application Publication
(10) Pub. No.: US 2018/0280184 A1
(43) Pub. Date: Oct. 4, 2018

(54) EXTERNAL FIXATION DEVICE FOR CERVICAL VERTEBRA

(71) Applicant: Xieping ZHU, Shanghai (CN)

(72) Inventor: Xieping ZHU, Shanghai (CN)

(21) Appl. No.: 15/790,083

(22) Filed: Oct. 23, 2017

(30) Foreign Application Priority Data
Mar. 28, 2017 (CN) 201720310370.4

(51) Int. Cl. A61F 5/055

(52) U.S. Cl. CPC A61F 5/055; A41D 13/0512

(57) ABSTRACT

An external device for fixation of cervical vertebrae includes a waistcoat-shaped main body. The waistcoat-shaped main body includes a left part, a right part and polyurethane slurry. The left part and the right part respectively includes of two layers of knitting cloth and a cavity between the two layers of knitting cloth. The left part and the right part are filled with the polyurethane slurry. The left part and the right part are respectively provided with corresponding adjustable connecting structures. The external device for fixation of cervical vertebrae rapidly shapes according to human bodies and keeps the cervical vertebrae of patients in a proper position.

### CROSS-REFERENCE TO RELATED APPLICATIONS

Pursuant to 35 U.S.C. § 119 and the Paris Convention Treaty, this application claims the benefit of Chinese Patent Application No. 201720310370.4 filed Mar. 28, 2017, the contents of which are incorporated herein by reference. Inquiries from the public to applicants or assignees concerning this document or the related applications should be directed to: Matthias Scholl P. C., Attn.: Dr. Matthias Scholl Esq., 245 First Street, 18th Floor, and Cambridge, Mass. 02142.

### BACKGROUND OF THE INVENTION

### Field of the Invention

The invention relates to an external device for fixation of cervical vertebrae.

### Description of the Related Art

For patients suffering from acute or chronic damage of cervical vertebrae or injuries of soft tissues around cervical vertebrae, a fixation device is needed for temporary fixation and protection of the injured parts. Conventionally, a non-surgical external fixation device adopts a cervical collar, a traction plate, a traction pillow or other protective devices for fixation. A tubular plaster or tubular high polymer bandage is difficult to use because its size is not adjustable with respect to the circumference of the area to be fixated and according to the amount of post-fracture swelling, and because it does not allow for air exchange and it makes the examination and treatment of the fractures difficult once applied.

### SUMMARY OF THE INVENTION

In view of the above-described problems, it is one objective of the invention to provide an external fixation device which can limit the six degrees of freedom of cervical vertebrae in terms of structures and materials. The external fixation device fits the figure of the user, is convenient for use and has a good fixation effect. The external fixation device effectively supports and fixes the cervical vertebrae, keeps the cervical vertebrae of patients in a proper position and shortens treatment and recovery cycles.

To achieve the above objectives, in accordance with one embodiment of the invention, there is provided an external device for fixation of cervical vertebrae which comprises a waistcoat-shaped main body having a hood. The waistcoat-shaped main body comprises a left part, a right part, and polyurethane slurry.

The left part and the right part respectively comprise two layers of knitting cloth and a cavity between the two layers of knitting cloth.

The left part comprises a left chest part, a left shoulder part, a left back part, a left neck part, a left chin part, a left occiput part, and an annual left forehead belt; the left neck part is connected to the left chest part and the left back part, the left chin part extends upwards from a front part of the left neck part; a left occiput part extends upwards from a back part of the left neck part; the annular left forehead belt extends outwards from the left side end of the upper part of the left occiput part; the left chest part is connected to the left back part through the left shoulder part, the underarm edge of the left chest part and the underarm edge of the left back part are respectively provided with a left underarm hook and loop, and the underarm edge of the left chest part is connected with the underarm edge of the left back part through the left underarm hook and loop so as to form a left arm hole for receiving a left arm of a user; the front part of the left neck part is a part of the left neck part connected to the left chest part; the back part of the left neck part is a part of the left neck part connected to the left back part; the left side end of the upper part of the left occiput part is the side end of the two side ends of the upper part of the left occiput part closer to the side end of the left shoulder line in terms of the horizontal distance; the underarm edge of the left chest part extends from a position corresponding to a left underarm of the user to a position corresponding to a waist line of the user; the underarm edge of the left back part extends from a position corresponding to the left underarm of the user to a position corresponding to the waist line of the user.

The right part comprises a right chest part, a right shoulder part, a right back part, a right neck part, a right chin part, a right occiput part, and an annular right forehead belt; the right neck part is connected to the right chest part and the right back part; the right chin part extends upwards from the front part of the right neck part; the right occiput part extends upwards from the back part of the right neck part; the annular right forehead belt extends outwards from the right side of the upper part of the right occiput part; the right chest part is connected to the right back part through the right shoulder part, the underarm edge of the right chest part and the underarm edge of the right back part are respectively provided with a right underarm hook and loop, and the underarm edge of the right chest part is connected to the underarm edge of the right back part through the right underarm hook and loop so as to form a right arm hole for receiving a right arm of the user; the front part of the right neck part is a part of the right neck part connected to the right chest part; the back part of the right neck part is a part of the right neck part connected to the right back part; the right side of the upper part of the right occiput part is the side end of the two side ends of the upper part of the right occiput part closer to the side end of the right shoulder line in terms of the horizontal distance; the underarm edge of the right chest part extends from a position corresponding to a right underarm of the user to a position corresponding to a waist line of the user; the underarm edge of the right back part extends from a position corresponding to the right underarm of the user to a position corresponding to the waist line of the user.

The lower edge of the left back part and the lower edge of the right back part are respectively provided with a filling port; a hook and loop is disposed on the filling port for closing or opening the filling port; the lower edge of the left back part or the right back part is an edge of the left back part or the right back part closest to the waist line of the use; the right side edge of the left chest part, the right side edge of the front part of the left neck part, the right side edge of the left chin part, the left side edge of the right chest part, the left side edge of the front part of the right neck part and the left side edge of the right chin part are respectively provided with a connecting piece of front central seam; the left chest part, the front part of the left neck part and the right side edge of the left chin part are respectively connected to the right chest part, the front part of the right neck part and the left side edge of the right chin part through the connecting piece of front central seam; the right side edge of the left chest part is an edge of the left chest part closest to the right chest part in a wearing state of the external device; the right side edge of the front part of the left neck part is an edge of the front part of the left neck part closest to the right neck part in the wearing state; the right side edge of the left chin part is an edge of the left chin part closest to the right chin part in the wearing state; the left side edge of the right chest part is an edge of the right chest part closest to the left chest part in the wearing state; the left side edge of the front part of the right neck part is an edge of the front part of the right neck part closest to the left neck part in the wearing state; the left side edge of the right chin part is an edge of the right chin part closest to the left chin part in the wearing state.

The right side edge of the left back part, the right side edge of the back part of the left neck part, the right side edge of the upper part of the left occiput part, the left side edge of the right back part, the left side edge of the back part of the right neck part and the left side edge of the upper part of the right occiput part are respectively provided with a connecting piece of back central seam; the left back part, the back part of the left neck part and the right side edge of the upper part of the left occiput part are respectively connected with the right back part, the back part of the right neck part and the left side edge of the upper part of the right occiput part through the connecting piece of back central seams; the right side edge of the left back part is an edge of the left back part closest to the right back part in the wearing state; the right side edge of the back part of the left neck part is an edge of the back part of the left neck part closest to the right neck part in the wearing state; the right side edge of the upper part of the left occiput part is an edge of the upper part of the left occiput part closest to the right occiput part in the wearing state; the left side edge of the right back part is an edge of the right back part closest to the left back part in the wearing state; the left side edge of the back part of the right neck part is an edge of the back part of the right neck part closest to the left neck part in the wearing state; the left side edge of the upper part of the right occiput part is an edge of the upper part of the right occiput part closest to the left occiput part.

The tail end of the annular left forehead belt and the tail end of the annular right forehead belt are respectively provided with a forehead hook and loop; the tail end of the annular left forehead belt is the end of the annular left forehead belt farthest from the left side end of the upper part of the left occiput part; the tail end of the annular right forehead belt is the end of the annular right forehead belt farthest from the right side end of the upper part of the right occiput part.

The cavity of the left part and the cavity of the right part are respectively filled with the polyurethane slurry through the filling port.

In a class of this embodiment, the cavity of the left part is arranged in the left chest part, the left shoulder part, the left back part, the left neck part, the left chin part and the left occiput part of the left part; the cavity of the right part is arranged in the right chest part, the right shoulder part, the right back part, the right neck part, the right chin part and the right occiput part of the right part. The cavities of the left chest part, the left shoulder part, the left back part, the left neck part, the left chin part and the left occiput part communicate with each other. The right chest part, the right shoulder part, the right back part, the right neck part, the right chin part and the right occiput part of the right part communicate with each other.

In a class of this embodiment, the tail ends of the annular left forehead belt and the annular right forehead belt are provided with seams, such that the cavity in the tail end of the annular left forehead belt does not communicate with the cavities in other parts of the left part; and the cavity in the tail end of the annular right forehead belt does not communicate with the cavities in other parts of the right part.

In a class of this embodiment, the underarm edge of the left chest part and the underarm edge of the left back part are provided with seams. Therefore, the cavities in the underarm edge of the left chest part and the underarm edge of the left back part are not connected with the cavities in other parts of the left part.

In a class of this embodiment, the underarm edge of the right chest part and the underarm edge of the right back part are provided with seams, such that the cavities in the underarm edge of the right chest part and the underarm edge of the right back part do not communicate with the cavities in other parts of the right part.

In a class of this embodiment, the connecting piece of front central seam and the connecting piece of back central seam are both zippers.

The advantages provided by the external fixation device are that: in the wearing state, the left and right parts are respectively filled with polyurethane slurry through the filling ports of the left and right back parts; the polyurethane slurry solidifies during the soft stage, and the device for fixation of cervical vertebrae closely wraps the upper body, the neck, the chin and the forehead of the user so as to precisely fix the cervical vertebrae and the head the user through the connecting piece of front central seam, the connecting piece of back central seam, the left underarm hook and loop, the right underarm hook and loop and the forehead hook and loop.

The external fixation device limits the six degrees of freedom of cervical vertebrae in terms of structures and materials. The external fixation device is rapidly shaped to closely wrap the upper body, the neck, the chin and the forehead of human body in the wearing state. The external fixation device is convenient in use and effectively supports and fixes cervical vertebrae, effectively limits the six degrees of freedom of cervical vertebrae, keeps the cervical vertebrae of patients in a proper position. The external fixation device can fully meet users' treatment demand, effectively reduce the clinical complications of users, consolidate a good restoration effect, promote the healing speed and quality and shorten treatment and recovery cycles.

### BRIEF DESCRIPTION OF THE DRAWINGS S

FIG. 1 is a stereograph of an external device for fixation of cervical vertebrae in according to one embodiment of the disclosure; and

FIG. 2 is a left view of an external device for fixation of cervical vertebrae in according to one embodiment of the disclosure.

### DETAILED DESCRIPTION OF THE EMBODIMENTS

The present invention will be further described with reference to the accompanying drawings.

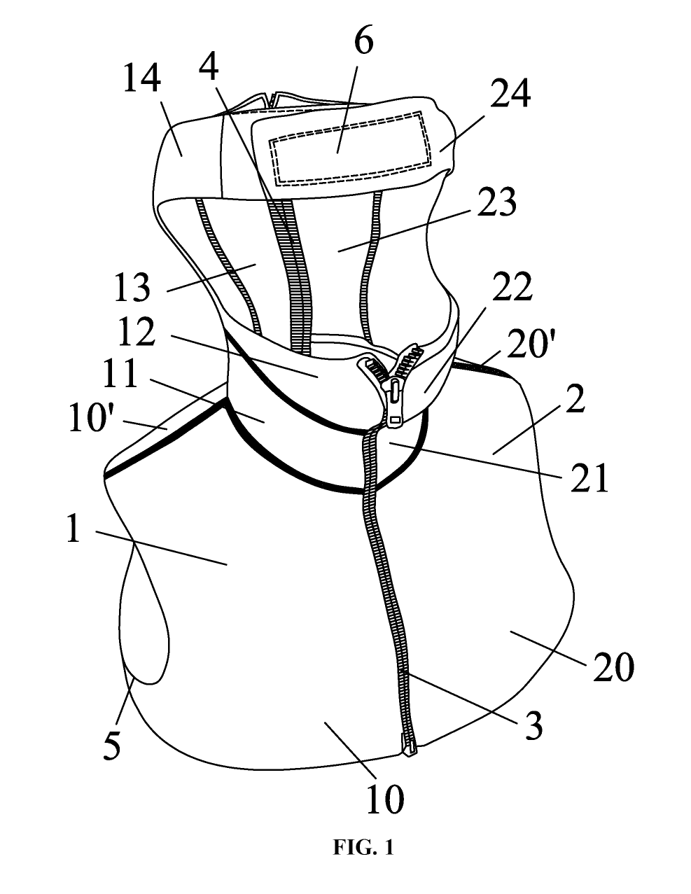

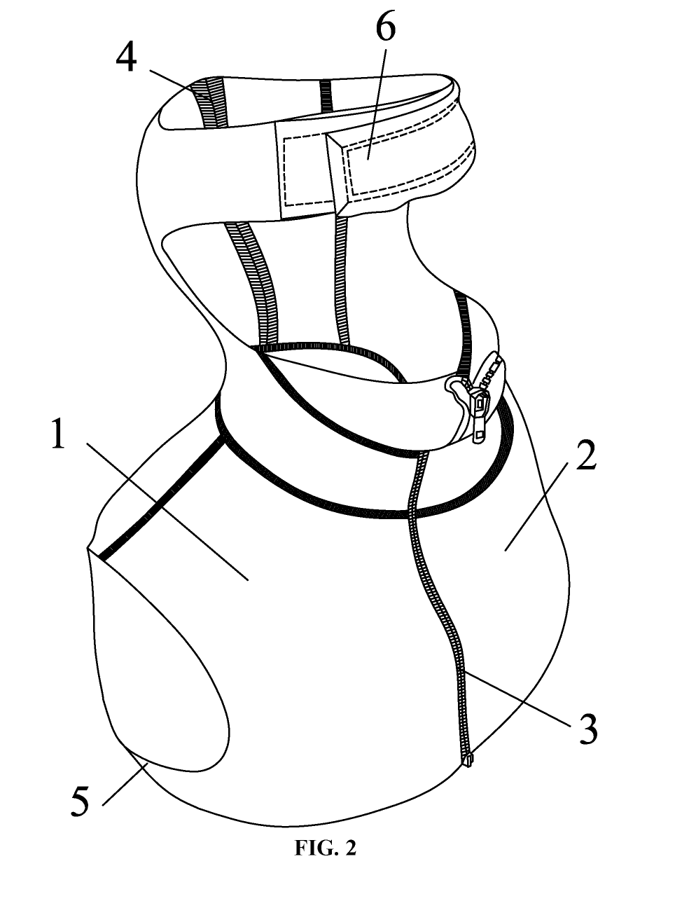

As shown in FIGS. 1 and 2, an external device for fixation of cervical vertebrae comprises a waistcoat-shaped main body having a hood. The waistcoat-shaped main body comprises a left part 1, a right part 2 and polyurethane slurry which is prepared by mixing polyurethane slurry A and polyurethane slurry B (the detailed technical scheme is disclosed in the U.S. Pat. No. 9,617,370).

The left part 1 comprises a left chest part 10, a left shoulder part, a left back part 10′ connected with the left chest part 10 through the left shoulder part, a left neck part 11 connected with the left chest part 10 and the left back part 10′, a left chin part 12 extending upwards from the front part of the left neck part 11, a left occiput part 13 extending upwards from the back part of the left neck part 11 and a annular left forehead belt 14 extending outwards from the left side end of the upper part of the left occiput part 13.

The right part 2 comprises a right chest part 20, a right shoulder part, a right back part 20′ connected with the right chest part 20 through the right shoulder part, a right neck part 21 connected with the right chest part 20 and the right back part 20′, the right chin part 22 extending upwards from the front part of the right neck part 21, the right occiput part 23 extending upwards from the back part of the right neck part 21 and a annular right forehead belt 24 extending outwards from the right side of the upper part of the right occiput part 23.

The left part 1 and the right part 2 respectively comprises two layers of knitting cloth and a cavity between the two layers of knitting cloth. The lower edge of the left back part 10 and the lower edge of the right back part 20′ are respectively provided with a filling port which is sewn with a hook and loop for closing the filling ports.

The right side edge of the left chest part 10, the right side edge of the front part of the left neck part 11, the right side edge of the left chin part 12, the left side edge of the right chest part 20, the left side edge of the front part of the right neck part 21 and the left side edge of the right chin part 22 are respectively provided with a connecting piece of front central seam 3. The connecting piece of front central seam 3 is a zipper.

The right side edge of the left back part 10′, the right side edge of the back part of the left neck part 11, the right side edge of the left occiput part 13, the left side edge of the right back part 20′, the left side edge of the back part of the right neck part 21 and the left side edge of the right occiput part 23 are respectively provided with a connecting piece of back central seam 4. The connecting piece of back central seam 4 is a zipper.

The underarm edge of the left chest part 10 and the underarm edge of the left back part 10′ are respectively provided with a left underarm hook and loop 5.

The underarm edge of the right chest part 20 and the underarm edge of the right back part 20′ are respectively provided with a right underarm hook and loop.

The tail end of the annular left forehead belt 14 and the tail end of the annular right forehead belt 24 are respectively provided with a forehead hook and loop 6.

The cavity of the left part and the cavity of the right part are respectively filled with the polyurethane slurry through the filling ports.

The external device for fixation of cervical vertebrae first fills the cavity of the left part 1 with the polyurethane slurry specially prepared by mixing polyurethane slurry A and polyurethane slurry B through the filling port of the left back part 10′ and fills the cavity of the right part 2 with the polyurethane slurry specially prepared by mixing polyurethane slurry A and polyurethane slurry B through the filling port of the right back part 20′. Then, the rolling and evening treatment of the polyurethane slurry is carried out on an operation platform. The polyurethane slurry solidifies. During the soft stage, through the connecting piece of front central seam 3, the connecting piece of back central seam 4, the left underarm hook and loop 5, the right underarm hook and loop and the forehead hook and loop 6, the device for fixation of cervical vertebrae closely wraps the upper body, the neck, the chin and the forehead of human body. Among them, the left neck part 11 and the right neck part 21 fit the neck of human body and act as splints to fit the patient's neck. Meanwhile, the annular left forehead belt 14 and the annular right forehead belt 24 wrap the head of human body through the forehead hook and loop 6 to put the patient's head in a proper position. The combination of the left chin part 12 and the right chin part 22 and the combination of the left occiput part 13 and the right occiput 23 can support the patient's head, effectively limit the six degrees of freedom of cervical vertebrae and keep the cervical vertebrae in a proper position.

The cavities in the tail end of the annular left forehead belt 14 and the tail end of the annular right forehead belt 24 do not communicate with other cavities through seams so that the tail end of the annular left forehead belt 14 and the tail end of the annular right forehead belt 24 are not filled with the polyurethane slurry so as to facilitate the connection of the forehead hook and loop 6. The cavities in the underarm edge of the left chest part 10 and the underarm edge of the left back part 10′ do not communicate with other cavities through seams so that the underarm edge of the left chest part 10 and the underarm edge of the left back part 10′ are not filled with the polyurethane slurry so as to facilitate the connection of the underarm hook and loop 5. The cavities in the underarm edge of the right chest part 20 and the underarm edge of the right back part 20′ do not communicate with other cavities through seams so that the underarm edge of the right chest part 20 and the underarm edge of the right back part 20′ are not filled with the polyurethane slurry so as to facilitate the connection of the right underarm hook and loop.

The external fixation device can limit the six degrees of freedom of cervical vertebrae in terms of structures and materials, is convenient in use, can be rapidly shaped according to the shape of human body, has a good fixation effect, can effective limit the six degrees of freedom of patients, fully meets patients' treatment demand and shortens treatment and recovery circles.

The definitions of the forward, backward, left, right, up and down directions of the invention are equal to the definitions of the forward, backward, left, right, up and down directions of human body. The horizontal and vertical directions of the invention refer to the directions which are horizontal and vertical with the ground.

While particular embodiments of the invention have been shown and described, it will be obvious to those skilled in the art that changes and modifications may be made without departing from the invention in its broader aspects, and therefore, the aim in the appended claims is to cover all such changes and modifications as fall within the true spirit and scope of the invention.

The invention claimed is:

1. An external device for fixation of cervical vertebrae, comprising:
a waistcoat-shaped main body; the waistcoat-shaped main body comprising a left part, a right part, and polyurethane slurry;
wherein:
the left part and the right part respectively comprise two layers of knitting cloth and a cavity between the two layers of knitting cloth;
the left part comprises a left chest part, a left shoulder part, a left back part, a left neck part, a left chin part, a left occiput part, and an annual left forehead belt;
the left neck part is connected to the left chest part and the left back part;
the left chin part extends upwards from a front part of the left neck part;
the left occiput part extends upwards from a back part of the left neck part;
the annular left forehead belt extends outwards from a left side end of an upper part of the left occiput part;
the left chest part is connected to the left back part through the left shoulder part;
an underarm edge of the left chest part and an underarm edge of the left back part are respectively provided with a left underarm hook and loop, and the underarm edge of the left chest part is connected to the underarm edge of the left back part through the left underarm hook and loop so as to form a left arm hole for receiving a left arm of a user;
the front part of the left neck part is a part of the left neck part connected to the left chest part;
the back part of the left neck part is a part of the left neck part connected to the left back part;
a left side end of the upper part of the left occiput part is a side end of two side ends of the upper part of the left occiput part closer to a side end of a left shoulder line in terms of the horizontal distance;
the underarm edge of the left chest part extends from a position corresponding to a left underarm of the user to a position corresponding to a waist line of the user; the underarm edge of the left back part extends from a position corresponding to the left underarm of the user to a position corresponding to the waist line of the user;
the right part comprises a right chest part, a right shoulder part, a right back part, a right neck part, a right chin part, a right occiput part, and an annular right forehead belt;
the right neck part is connected to the right chest part and the right back part;
the right chin part extends upwards from a front part of the right neck part;
the right occiput part extends upwards from a back part of the right neck part;
the annular right forehead belt extends outwards from a right side of an upper part of the right occiput part;
the right chest part is connected to the right back part through the right shoulder part;
an underarm edge of the right chest part and an underarm edge of the right back part are respectively provided with a right underarm hook and loop, and the underarm edge of the right chest part is connected to the underarm edge of the right back part through the right underarm hook and loop so as to form a right arm hole for receiving a right arm of the user;
the front part of the right neck part is a part of the right neck part connected to the right chest part;
the back part of the right neck part is a part of the right neck part connected to the right back part;
the right side of the upper part of the right occiput part is a side end of two side ends of the upper part of the right occiput part closer to a side end of a right shoulder line in terms of the horizontal distance;
the underarm edge of the right chest part extends from a position corresponding to a right underarm of the user to a position corresponding to a waist line of the user; the underarm edge of the right back part extends from a position corresponding to the right underarm of the user to a position corresponding to the waist line of the user;
the lower edge of the left back part and the lower edge of the right back part are respectively provided with a filling port; a hook and loop is disposed on the filling port for closing or opening the filling port;
the lower edge of the left back part is an edge of the left back part closest to the waist line of the user; the lower edge of the right back part is an edge of the right back part closest to the waist line of the user;
a right side edge of the left chest part, a right side edge of the front part of the left neck part, a right side edge of the left chin part, a left side edge of the right chest part, a left side edge of the front part of the right neck part and a left side edge of the right chin part are respectively provided with a connecting piece of front central seam;
the left chest part, the front part of the left neck part and the right side edge of the left chin part are respectively connected to the right chest part, the front part of the right neck part and the left side edge of the right chin part through the connecting piece of front central seam;
the right side edge of the left chest part is an edge of the left chest part closest to the right chest part in a wearing state of the external device; the right side edge of the front part of the left neck part is an edge of the front part of the left neck part closest to the right neck part in the wearing state; the right side edge of the left chin part is an edge of the left chin part closest to the right chin part in the wearing state; the left side edge of the right chest part is an edge of the right chest part closest to the left chest part in the wearing state; the left side edge of the front part of the right neck part is an edge of the front part of the right neck part closest to the left neck part in the wearing state; the left side edge of the right chin part is an edge of the right chin part closest to the left chin part in the wearing state;
a right side edge of the left back part, a right side edge of the back part of the left neck part, a right side edge of the upper part of the left occiput part, a left side edge of the right back part, a left side edge of the back part of the right neck part and a left side edge of the upper part of the right occiput part are respectively provided with a connecting piece of back central seam;
the left back part, the back part of the left neck part and the right side edge of the upper part of the left occiput part are respectively connected to the right back part, the back part of the right neck part and the left side edge of the upper part of the right occiput part through the connecting piece of back central seam;
the right side edge of the left back part is an edge of the left back part closest to the right back part in the wearing state; the right side edge of the back part of the left neck part is an edge of the back part of the left neck part closest to the right neck part in the wearing state; the right side edge of the upper part of the left occiput part is an edge of the upper part of the left occiput part closest to the right occiput part in the wearing state; the left side edge of the right back part is an edge of the right back part closest to the left back part in the wearing state; the left side edge of the back part of the right neck part is an edge of the back part of the right neck part closest to the left neck part in the wearing state; the left side edge of the upper part of the right occiput part is an edge of the upper part of the right occiput part closest to the left occiput part;
an tail end of the annular left forehead belt and a tail end of the annular right forehead belt are respectively provided with a forehead hook and loop; the tail end of the annular left forehead belt is the end of the annular left forehead belt farthest from the left side end of the upper part of the left occiput part; the tail end of the annular right forehead belt is the end of the annular right forehead belt farthest from the right side end of the upper part of the right occiput part; and
the cavity of the left part and the cavity of the right part are respectively filled with the polyurethane slurry through the filling port.

2. The device of claim 1, wherein the connecting piece of front central seam and the connecting piece of back central seam are both zippers.
